(19) United States
(12) Patent Application Publication
(10) Pub. No.: US 2005/0113882 A1
(43) Pub. Date: May 26, 2005

(54) Electrical stimulation system, lead, and method providing reduced neuroplasticity effects

(75) Inventors: Tracy L. Cameron, Toronto (CA); Christopher G. Chavez, McKinney, TX (US)

Correspondence Address: BAKER BOTTS L.L.P., 2001 ROSS AVENUE, 6TH FLOOR, DALLAS, TX, 75201-2980, US

(73) Assignee: Advanced Neuromodulation Systems, Inc.

(21) Appl. No.: 10/994,008

(22) Filed: Nov. 18, 2004

Related U.S. Application Data
(60) Provisional application No. 60/523,710, filed on Nov. 20, 2003.

(51) Int. Cl. A61N 1/18

(52) U.S. Cl. 607/45

(57) ABSTRACT

According to one aspect, an electrical stimulation system provides reduced neuroplasticity effects in a person's nerve tissue. The system includes an electrical stimulation lead adapted for implantation into the person's body for electrical stimulation of target nerve tissue. The lead includes a number of electrodes adapted to be positioned near the target nerve tissue and to deliver electrical stimulation energy to the target nerve tissue. The system also includes a stimulation source connectable to the lead and operable to generate signals for transmission to the electrodes of the lead to cause the electrodes to deliver electrical stimulation energy to the target nerve tissue to reduce neuroplasticity effects.

### RELATED APPLICATIONS

This application claims the benefit under 35 U.S.C. § 119(e) of U.S. Provisional Application Ser. No. 60/523,710, filed Nov. 20, 2003.

### TECHNICAL FIELD OF THE INVENTION

This invention relates generally to electrical stimulation of nerve tissue and in particular to an electrical stimulation system, lead, and method providing reduced neuroplasticity effects.

### BACKGROUND

Many people experience adverse conditions associated with functions of the cortex, the thalamus, and other brain structures. Such conditions have been treated effectively using electrical stimulation systems incorporating leads with electrodes implanted in the brain near a target tissue. According to one technique, a set of efficacious electrical stimulation parameters are determined, the set of parameters is entered into the system, and the system is used to electrically stimulate the brain according to the set of parameters to treat the condition. Typically, an implanted signal generator transmits signals to the implanted lead according to the set of parameters and, in response to the signals, the electrodes of the implanted lead deliver electrical energy to the target tissue to treat the condition.

Although electrical simulation of the brain is often an effective treatment, the efficacy of the treatment associated with a particular set of stimulation parameters often decreases in time due to neuroplasticity of the brain. Neuroplasticity refers to the ability of the brain to dynamically reorganize itself in response to certain stimuli to form new neural connections. This allows the neurons in the brain to compensate for injury or disease and adjust their activity in response to new situations or changes in their environment. With respect to electrical stimulation, the reduction in efficacy due to neuroplasticity often occurs after just a few weeks of treatment. In order to regain the same efficacy, a new set of efficacious electrical stimulation parameters must be determined, the new set of parameters must be entered into the system, and the system is again used to electrically stimulate the brain according to the new set of parameters to continue to treat the condition. This results in the additional time and expense associated with a return visit to the treating physician for determining and entering the new set of parameters. Especially where treatment is to continue over a relatively long period of time, such as months or years, this additional time and expense poses a significant drawback.

### SUMMARY OF THE INVENTION

The electrical stimulation system, lead, and method of the present invention may reduce or eliminate certain problems and disadvantages associated with prior techniques for electrically stimulating the brain.

According to one aspect, an electrical stimulation system provides reduced neuroplasticity effects in a person's nerve tissue. The system includes an electrical stimulation lead adapted for implantation into the person's body for electrical stimulation of target nerve tissue. The lead includes a number of electrodes adapted to be positioned near the target nerve tissue and to deliver electrical stimulation energy to the target nerve tissue. The system also includes a stimulation source connectable to the lead and operable to generate signals for transmission to the electrodes of the lead to cause the electrodes to deliver electrical stimulation energy to the target nerve tissue to reduce neuroplasticity effects.

Particular embodiments of the present invention may provide one or more technical advantages. According to the present invention, an electrical stimulation system is used to provide electrical stimulation of the brain to reduce neuroplasticity effects. For example, in certain situations, the onset of neuroplasticity effects associated with therapeutic electrical stimulation of the brain may be prevented, delayed, or otherwise reduced. As a result, in certain embodiments, the efficacy period associated with a particular set of stimulation parameters may be extended. This may help prevent the additional time and expense associated with one or more return visits to the treating physician for determining and entering new sets of efficacious parameters. Especially where the treatment is to continue over a relatively long period of time, such as a number of months or years, avoiding this additional time and expense may provide a significant advantage. As another example, in other situations, the further development of neuroplasticity effects already in existence due to injury or disease may be prevented, delayed, or otherwise reduced, or such pre-existing neuroplasticity effects may be reversed in whole or in part. As a result, in certain embodiments, pain or other conditions resulting from such pre-existing neuroplasticity effects may be prevented from progressing further, may be reduced, or may even be eliminated. Certain embodiments may provide all, some, or none of these advantages. Certain embodiments may provide one or more other advantages, one or more of which may be apparent to those skilled in the art from the figures, descriptions, and claims included herein.

### BRIEF DESCRIPTION OF THE DRAWINGS

For a more complete understanding of the present invention and advantages thereof, reference is now made to the following description taken in conjunction with the accompanying drawings, in which:

FIGS. 1A-1B illustrate example electrical stimulation systems providing reduced neuroplasticity effects in a person's brain;

FIG. 2 illustrates example steps that may be used to implant an example electrical stimulation system into a person for electrical stimulation of the person's brain;

FIGS. 3A-3I illustrate example electrical stimulation leads that may be used to provide reduced neuroplasticity effects in a person's brain;

FIG. 4 illustrates an example stimulation set;

FIG. 5 illustrates a number of example stimulation programs, each of which includes a number of stimulation sets; and

FIG. 6 illustrates example execution of a sequence of stimulation sets within an example stimulation program.

### DESCRIPTION OF EXAMPLE EMBODIMENTS

According to the present invention, an electrical stimulation system is used to provide electrical stimulation of the brain to reduce neuroplasticity effects. For example, according to the present invention, the onset of neuroplasticity effects associated with therapeutic electrical stimulation of the brain may be prevented, delayed, or otherwise reduced. As a result, the efficacy period associated with a particular set of stimulation parameters may be extended. This may help prevent the additional time and expense associated with one or more return visits to the treating physician for determining and entering new sets of efficacious parameters. Especially where treatment is to continue over a relatively long period of time, such as months or years, avoiding this additional time and expense may provide a significant advantage. As another example, the further development of neuroplasticity effects already in existence due to injury or disease may be prevented, delayed, or otherwise reduced, or such pre-existing neuroplasticity effects may be reversed in whole or in part. In one embodiment, the nature of the neuroplasticity reducing electrical stimulation may be varied more or less continually, in a predetermined or randomized manner, to prevent, delay, or otherwise reduce the ability of the brain to adapt to the neuroplasticity reducing electrical stimulation and dynamically reorganize itself accordingly. In a more particular embodiment, where the neuroplasticity reducing electrical stimulation is provided concurrently with therapeutic electrical stimulation, the neuroplasticity reducing electrical stimulation may be randomized or otherwise varied about the therapeutic electrical stimulation to achieve this result. In essence, the randomized or otherwise varied neuroplasticity reducing electrical stimulation makes it more difficult for the brain to dynamically reorganize itself to overcome the effects of the therapeutic electrical stimulation.

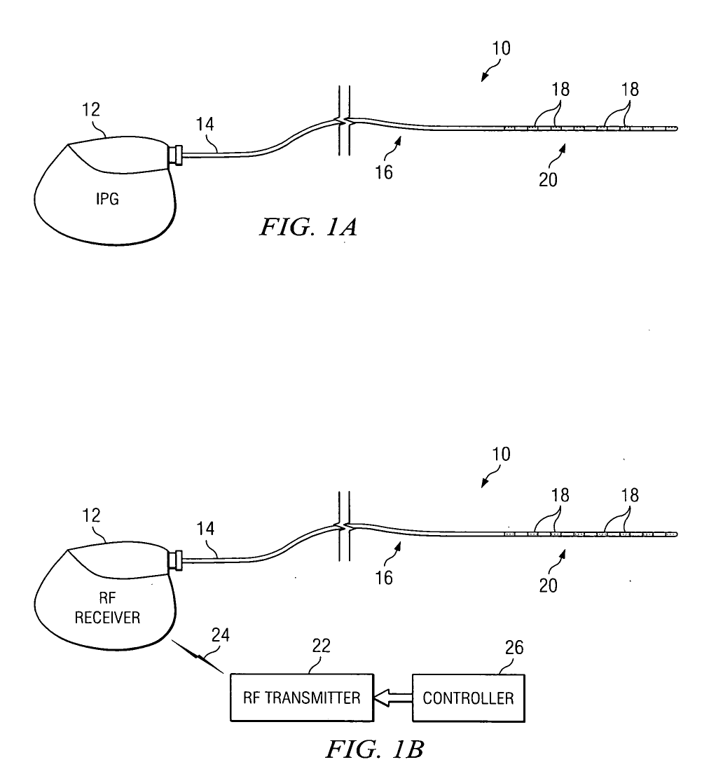

FIGS. 1A-1B illustrate example electrical stimulation systems 10 used to provide reduced neuroplasticity effects associated with therapeutic electrical stimulation of the brain, the spinal cord, or a peripheral nerve or due to previous injury or disease of the brain, the spinal cord, or a peripheral nerve. Stimulation system 10 generates and applies a stimulus to a target area of the brain, spinal cord, or peripheral nerve. For example, a target area may be an area of the brain located in the cortex or, as a more particular example, in the primary auditory cortex to treat tinnitus. In general terms, stimulation system 10 includes an implantable electrical stimulation source 12 and an implantable electrical stimulation lead 14 for applying the stimulation signal to targeted nerve tissue. In operation, both of these primary components are implanted in the person's body. Stimulation source 12 is coupled to a connecting portion 16 of electrical stimulation lead 14. Stimulation source 12 controls the electrical signals transmitted to electrodes 18 located on a stimulating portion 20 of electrical stimulation lead 14, located adjacent the target brain tissue, according to suitable signal parameters (e.g., duration, intensity, frequency, etc.). A doctor, the patient, or another user of stimulation source 12 may directly or indirectly input signal parameters for controlling the nature of the electrical stimulation provided.

In one embodiment, as shown in FIG. 1A, stimulation source 12 includes an implantable pulse generator (IPG). An example IPG may be one manufactured by Advanced Neuromodulation Systems, Inc., such as the Genesis® System, part numbers 3604, 3608, 3609, and 3644. In another embodiment, as shown in FIG. 1B, stimulation source 12 includes an implantable wireless receiver. An example wireless receiver may be one manufactured by Advanced Neuromodulation Systems, Inc., such as the Renew® System, part numbers 3408 and 3416. The wireless receiver is capable of receiving wireless signals from a wireless transmitter 22 located external to the person's body. The wireless signals are represented in FIG. 1B by wireless link symbol 24. A doctor, the patient, or another user of stimulation source 12 may use a controller 26 located external to the person's body to provide control signals for operation of stimulation source 12. Controller 26 provides the control signals to wireless transmitter 22, wireless transmitter 22 transmits the control signals and power to the wireless receiver of stimulation source 12, and stimulation source 12 uses the control signals to vary the signal parameters of electrical signals transmitted through electrical stimulation lead 14 to the stimulation site. An example wireless transmitter 122 may be one manufactured by Advanced Neuromodulation Systems, Inc., such as the Renew® System, part numbers 3508 and 3516.

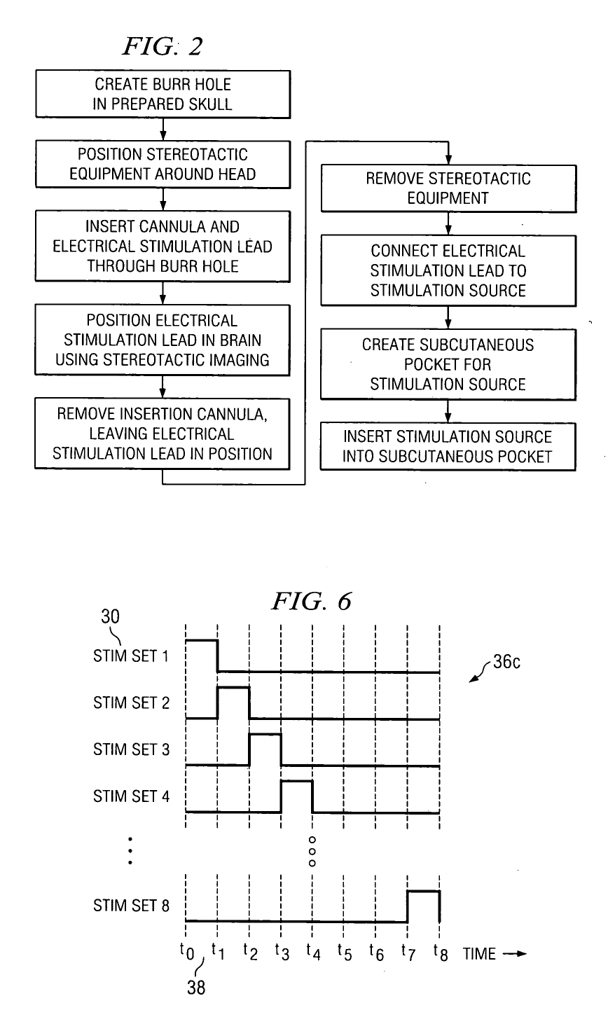

FIG. 2 illustrates example steps that may be used to implant an example stimulation system 10 into a person for electrical stimulation of the person's nerve tissue, for example, electrical stimulation of the brain, the spinal cord, or a peripheral nerve. For example, the cortex or, as a more particular example, the primary auditory cortex to treat tinnitus. In one embodiment, for stimulation of a person's brain, the skull is first prepared by exposing the skull and creating a burr hole in the skull. A burr hole cover may be seated within the burr hole and fixed to the scalp or skull. Stereotactic equipment suitable to aid in placement of an electrical stimulation lead 14 in the brain may be positioned around the head. Typically, an insertion cannula for electrical stimulation lead 14 is inserted through the burr hole into the brain, but a cannula is not required. For example, a hollow needle may provide the cannula. The cannula and electrical stimulation lead 14 may be inserted together or lead 14 may be inserted through the cannula after the cannula has been inserted. Using stereotactic imaging guidance or otherwise, electrical stimulation lead 14 is precisely positioned in the brain adjacent the target brain tissue, for example, target brain tissue in the cortex or, as a more particular example, in the primary auditory cortex to treat tinnitus.

Once electrical stimulation lead 14 has been positioned in the brain, lead 14 is uncoupled from any stereotactic equipment present, and the cannula and stereotactic equipment are removed. Where stereotactic equipment is used, the cannula may be removed before, during, or after removal of the stereotactic equipment. Connecting portion 16 of electrical stimulation lead 14 is laid substantially flat along the skull. Where appropriate, any burr hole cover seated in the burr hole may be used to secure electrical stimulation lead 14 in position and possibly to help prevent leakage from the burr hole and entry of contaminants into the burr hole. Example burr hole covers that may be appropriate in certain embodiments are illustrated and described in copending U.S. application Ser. Nos. 10/______ and 10/______, both filed November ______, 2003 and entitled “Electrical Stimulation System and Associated Apparatus for Securing an Electrical Stimulation Lead in Position in a Person's Brain” (Attorney's Docket 065274.0113 and 065274.0120).

Once electrical stimulation lead 14 has been inserted and secured, connecting portion 16 of lead 14 extends from the lead insertion site to the implant site at which stimulation source 12 is implanted. The implant site is typically a subcutaneous pocket formed to receive and house stimulation source 12. The implant site is usually positioned a distance away from the insertion site, such as near the buttocks or another place in the torso area. Once all appropriate components of stimulation system 10 are implanted, these components may be subject to mechanical forces and movement in response to movement of the person's body. A doctor, the patient, or another user of stimulation source 12 may directly or indirectly input signal parameters for controlling the nature of the electrical stimulation provided.

Although example steps are illustrated and described, the present invention contemplates two or more steps taking place substantially simultaneously or in a different order. In addition, the present invention contemplates using methods with additional steps, fewer steps, or different steps, so long as the steps remain appropriate for implanting an example stimulation system 10 into a person for electrical stimulation of the person's brain.

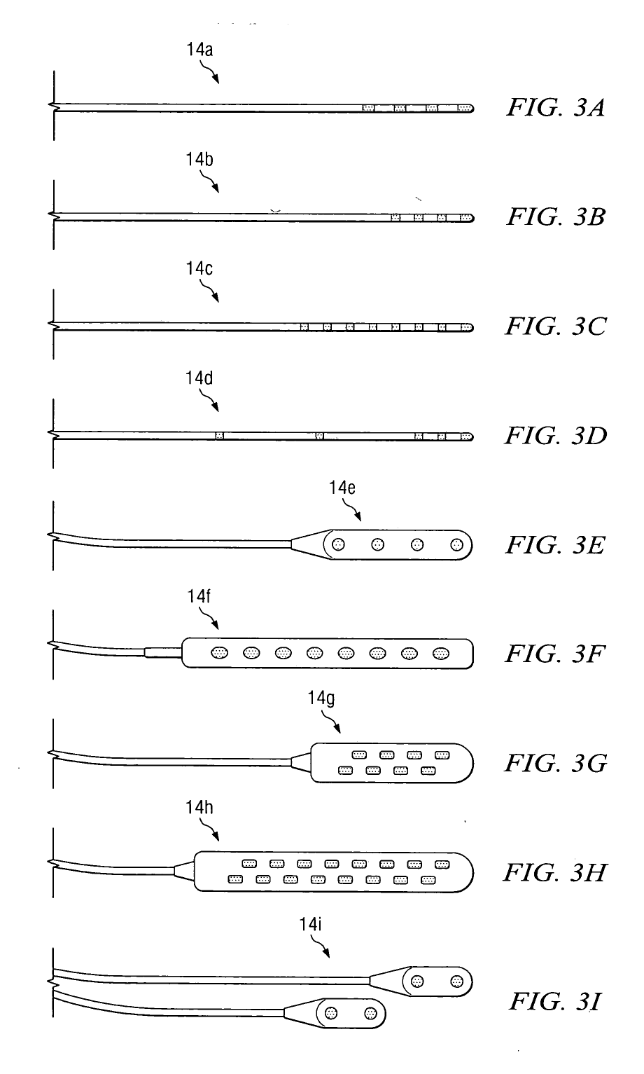

FIGS. 3A-3I illustrate example electrical stimulation leads 14 that may be used to provide reduced neuroplasticity effects in a person's brain, for example, associated with therapeutic electrical stimulation of the brain or due to previous injury or disease. As described above, each of the one or more leads 14 incorporated in stimulation system 10 includes one or more electrodes 18 adapted to be positioned near the target brain tissue and used to deliver electrical stimulation energy to the target brain tissue in response to electrical signals received from stimulation source 12. A percutaneous lead 14, such as example leads 14a-d, includes one or more circumferential electrodes 18 spaced apart from one another along the length of lead 14. Circumferential electrodes 18 emit electrical stimulation energy generally radially in all directions. A laminotomy or paddle style lead 14, such as example leads 14e-i, includes one or more directional electrodes 18 spaced apart from one another along one surface of lead 14. Directional electrodes 18 emit electrical stimulation energy in a direction generally perpendicular to the surface of lead 14 on which they are located. Although various types of leads 14 are shown as examples, the present invention contemplates stimulation system 10 including any suitable type of lead 14 in any suitable number. For example, unilateral stimulation of the brain is typically accomplished using a single lead 14 implanted in one side of the brain, while bilateral stimulation of the brain is typically accomplished using two leads 14 implanted in opposite sides of the brain.

In general, the cortex of a person's brain functions to provide a person with a representation of the external environment to allow the person to function effectively in that environment. The cortex includes frontal, parietal, occipital, and temporal regions that are each generally associated with particular functions.

The frontal cortex is generally associated with control of motor abilities and includes what is commonly referred to as the primary motor cortex. The frontal cortex also includes a region referred to as the prefrontal cortex that receives sensory information of multiple types, including autonomic sensory information from the internal organs, and is considered important for guiding behavior based on memory, translating ideas into words, and other functions. The parietal cortex is generally associated with sensory perception of the external environment and includes what is commonly referred to as the primary somatosensory cortex. The parietal cortex is also considered important for integrating sensory information of multiple types, for example, the ability to recognize the identity of a friend and imagine his face based only on the sound of his voice. The occipital cortex is generally associated with processing light and includes what is commonly referred to as the primary visual cortex. The temporal cortex is generally associated with processing sound and includes what is commonly referred to as the primary auditory cortex. The temporal cortex is also considered important for language comprehension, translation of words into speech, sensing balance and equilibrium, and certain complex aspects of vision. The above are provided merely as examples and are not intended to represent a full listing of the many functions associated with regions of the cortex, many of which may interact and overlap in complex ways to provide these functions.

Stimulation system 10 may be used to electrically stimulate and thus provide reduced neuroplasticity effects in the cortex (such as in the primary auditory cortex to treat tinnitus), the thalamus (which among other functions provides a center for routing certain types of incoming sensory information to higher level nerve centers in the cortex), or any other suitable target brain tissue. For example, where therapeutic electrical stimulation is directed to the primary somatosensory cortex for pain relief, stimulation system 10 may be used to apply additional electrical stimulation to the primary somatosensory cortex to reduce neuroplasticity effects associated with the therapeutic electrical stimulation. As another example, where therapeutic electrical stimulation is directed to the primary auditory cortex for tinnitus relief, stimulation system 10 may be used to apply additional electrical stimulation to the primary auditory cortex to reduce neuroplasticity effects associated with the therapeutic electrical stimulation.

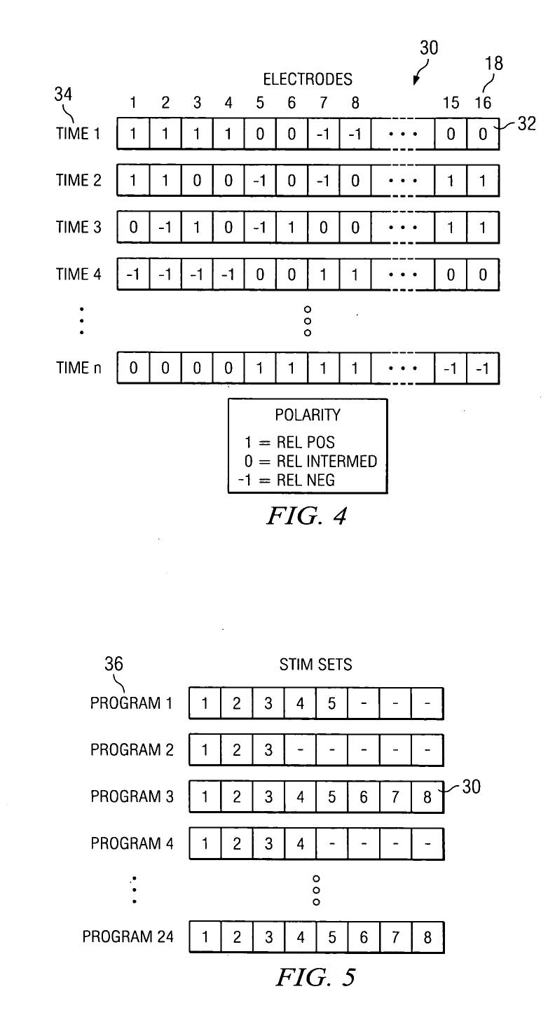

FIG. 4 illustrates an example stimulation set 30. One or more stimulation sets 30 may be provided, each stimulation set 30 specifying a number of stimulation parameters for the stimulation set 30. For example, as described more fully below with reference to FIGS. 5-6, multiple stimulation sets 30 may be executed in an appropriate sequence according to a pre-programmed stimulation program.

Stimulation parameters for a stimulation set 30 may include an amplitude, a frequency, phase information, and a pulse width for each of a series of stimulation pulses that electrodes 18 are to deliver to the target brain tissue during a time interval during which stimulation set 30 is executed, along with a polarity 32 for each electrode 18 within each stimulation pulse. Stimulation parameters may also include a pulse shape, for example, biphasic cathode first, biphasic anode first, or any other suitable pulse shape. One or more stimulation parameters for a stimulation set 30 may be randomized or otherwise varied in any suitable manner within the time interval in which stimulation set 30 is executed, spanning one or more stimulation pulses within each stimulation pulse. For example, instead of or in addition to randomizing or otherwise varying polarities 32 for electrodes 18 as described below, the amplitude, frequency, phase information, and pulse width may be randomized or otherwise varied within predetermined ranges, singly or in any suitable combination, within each stimulation pulse. As another example, instead of or in addition to randomizing or otherwise varying polarities 32 for electrodes 18 over multiple stimulation pulses as described more fully below, the amplitude, frequency, phase information, and pulse width may be randomized or otherwise varied within predetermined ranges, singly or in any suitable combination, over multiple stimulation pulses, where the combination of stimulation parameters is substantially constant within each stimulation pulse but different for successive stimulation pulses. As described above, such randomization or other variation of stimulation parameters for a stimulation set 30 may reduce the ability of the brain to adapt to the neuroplasticity reducing electrical stimulation and dynamically reorganize itself to overcome the effects of the neuroplasticity reducing stimulation.

The polarity for an electrode 18 at a time 34 beginning a corresponding stimulation pulse or sub-interval within a stimulation pulse may be a relatively positive polarity 32, a relatively negative polarity 32, or an intermediate polarity 32 between the relatively positive polarity 32 and relatively negative polarity 32. For example, the relatively positive polarity 32 may involve a positive voltage, the relatively negative polarity 32 may involve a negative voltage, and the relatively intermediate polarity 32 may involve a zero voltage (i.e. “high impedance”). As another example, the relatively positive polarity 32 may involve a first negative voltage, the relatively negative polarity 32 may involve a second negative voltage more negative than the first negative voltage, and the relatively intermediate polarity 32 may involve a negative voltage between the first and second negative voltages. The availability of three distinct polarities 32 for an electrode 18 may be referred to as “tri-state” electrode operation. The polarity 32 for each electrode 18 may change for each of the sequence of times 34 corresponding to stimulation pulses or to sub-intervals within a stimulation pulse according to the stimulation parameters specified for the stimulation set 30. For example, as is illustrated in FIG. 4 for an example stimulation set 30 for a lead 14 with sixteen electrodes 18, the polarities 32 of the sixteen electrodes 18 may change for each of the sequence of times 34. In the example of FIG. 4, a relatively positive polarity 32 is represented using a “1,” a relatively intermediate polarity 32 is represented using a “0,” and a relatively negative polarity 32 is represented using a “−1,” although any suitable values or other representations may be used. The polarity 32 for each electrode 18 may change in a predetermined or randomized manner, randomized changes possibly being more effective for reasons described above.

Where stimulation system 10 provides therapeutic electrical stimulation in addition to electrical stimulation to reduce neuroplasticity effects associated with the therapeutic electrical stimulation, each stimulation pulse or sub-interval within a stimulation pulse may be particular to the stimulation being provided; that is, either to therapeutic electrical stimulation or to neuroplasticity reducing electrical stimulation. For example, one or more stimulation pulses or sub-intervals may be designed to provide therapeutic electrical stimulation and one or more other stimulation pulses or sub-intervals may be designed to reduce neuroplasticity effects. In this case, the therapeutic stimulation pulses or sub-intervals and neuroplasticity reducing stimulation pulses or sub-intervals may be arranged temporally in any suitable manner. A therapeutic stimulation pulse or sub-interval may be separated from a successive therapeutic stimulation pulse or sub-interval by any number of neuroplasticity reducing stimulation pulses or sub-intervals and this number may be the same between each pair of therapeutic stimulation pulses or sub-intervals or may vary between each pair of therapeutic stimulation pulses or sub-intervals in a predetermined or randomized manner. As another example, one or more stimulation pulses or sub-intervals may be designed to concurrently provide both therapeutic and neuroplasticity reducing electrical stimulation.

Similarly where stimulation system 10 provides therapeutic electrical stimulation in addition to electrical stimulation to reduce neuroplasticity effects associated with the therapeutic electrical stimulation, each stimulation set 30 may be particular to either the therapeutic electrical stimulation or the neuroplasticity reducing electrical stimulation. For example, one or more stimulation sets 30 may be designed to provide therapeutic electrical stimulation and one or more other stimulation sets 30 may be designed to reduce neuroplasticity effects. In this case, the therapeutic stimulation sets 30 and neuroplasticity reducing stimulation sets 30 may be arranged temporally in any suitable manner. A therapeutic stimulation set 30 may be separated from a successive therapeutic stimulation set 30 by any number of neuroplasticity reducing stimulation sets 30 and this number may be the same between each pair of therapeutic stimulation sets 30 or may vary between each pair of therapeutic stimulation sets 30 in a predetermined or randomized manner. As another example, one or more stimulation sets 30 may be designed to concurrently provide both therapeutic and neuroplasticity reducing electrical stimulation.

In addition, the amplitude, frequency, phase information, or pulse width for a stimulation set 30 may be particular to the stimulation being provided. For example, therapeutic electrical stimulation may be provided using higher amplitude electrical energy than is used for neuroplasticity reducing electrical stimulation. In this case, the neuroplasticity reducing electrical stimulation may be substantially or totally imperceptible to the patient (i.e. below a perceptibility threshold where therapeutic electrical stimulation is provided for pain relief). Alternatively, neuroplasticity reducing electrical stimulation may be provided using the same or a higher amplitude electrical energy than is used for therapeutic electrical stimulation (i.e. at or above the perceptibility threshold where therapeutic electrical stimulation is provided for pain relief).

FIG. 5 illustrates a number of example stimulation programs 36, each including a number of stimulation sets 30. One or more simulation programs 36 are set up to provide reduced neuroplasticity effects, for example, associated with electrical stimulation of the brain or due to previous injury or disease. As described above, each stimulation set 30 specifies a number of stimulation parameters for the stimulation set 30. In one embodiment, within each stimulation program 36, stimulation system 10 consecutively executes the sequence of one or more stimulation sets 30 associated with stimulation program 36. The sequence may be executed only once, repeated a specified number of times, or repeated an unspecified number of times within a specified time period. For example, as is illustrated in FIG. 6 for the third example stimulation program 36c including eight stimulation sets 30, each of the eight stimulation sets 30 is consecutively executed in sequence. Although the time intervals 38 (t₁-t₀, t₂-t₁, etc.) during which the stimulation sets 30 are executed are shown as being equal, the present invention contemplates a particular stimulation set 30 being executed over a different time interval 38 than one or more other stimulation sets 30 according to particular needs. One or more stimulation sets 30 within at least one stimulation program 36 are set up to provide reduced neuroplasticity effects, for example, associated with electrical stimulation of the brain or due to previous injury or disease.

Although stimulation system 10 is illustrated by way of example as accommodating up to twenty-four stimulation programs 36 each including up to eight stimulation sets 30, the present invention contemplates any appropriate number of stimulation programs 36 each including any appropriate number of stimulation sets 30. For example, in a very simple case, a single stimulation program 36 may include a single stimulation set 30, whereas in a very complex case more than twenty-four stimulation programs 36 may each include more than eight stimulation sets 30.

In one embodiment, stimulation system 10 executes only a single stimulation program 36 in response to user selection of that stimulation program for execution. In another embodiment, during a stimulation period, stimulation system 10 executes a sequence of pre-programmed stimulation programs 36 for each lead 14 until the stimulation period ends. Depending on the length of the stimulation period and the time required to execute a sequence of stimulation programs 36, the sequence may be executed one or more times. For example, the stimulation period may be defined in terms of a predetermined number of cycles each involving a single execution of the sequence of stimulation programs 36, the sequence of stimulation programs 36 being executed until the predetermined number of cycles has been completed. As another example, the stimulation period may be defined in terms of time, the sequence of stimulation programs 36 being executed until a predetermined time interval has elapsed or the patient or another user manually ends the stimulation period. Although a sequence of stimulation programs 36 is described, the present invention contemplates a single stimulation program being executed one or more times during a stimulation period according to particular needs. Furthermore, the present invention contemplates each stimulation program 36 being executed substantially immediately after execution of a previous stimulation program 36 or being executed after a suitable time interval has elapsed since completion of the previous stimulation program 36. Where stimulation system 10 includes multiple leads 14, stimulation programs 36 for a particular lead 14 may be executed substantially simultaneously as stimulation programs 36 for one or more other leads 14, may be alternated with stimulation programs 36 for one or more other leads 14, or may be arranged in any other suitable manner with respect to stimulation programs 36 for one or more other leads 14.

Where stimulation system 10 provides therapeutic electrical stimulation in addition to electrical stimulation to reduce neuroplasticity effects, each stimulation program 36 may be particular to either the therapeutic electrical stimulation or the neuroplasticity reducing electrical stimulation. For example, one or more stimulation programs 36 may be designed to provide therapeutic electrical stimulation and one or more other stimulation programs 36 may be designed to reduce neuroplasticity effects. In this case, the therapeutic stimulation programs 36 and the neuroplasticity reducing stimulation programs 36 may be arranged temporally in any manner. A therapeutic stimulation program 36 may be separated from a successive therapeutic stimulation program 36 by any number of neuroplasticity reducing stimulation programs 36 and this number may be the same between each pair of therapeutic stimulation programs 36 or may vary between each pair of therapeutic stimulation programs 36 in a predetermined or randomized manner. As another example, one or more stimulation programs 36 may be set up to concurrently provide both therapeutic and neuroplasticity reducing electrical stimulation.

In general, each stimulation program 36 may, but need not necessarily, be set up for electrical stimulation of different target brain tissue. As an example, where therapeutic electrical stimulation of the primary motor cortex is desired, one or more stimulation programs 36 may be set up for therapeutic electrical stimulation of target brain tissue in the primary auditory cortex and one or more other stimulation programs 36 may be set up for electrical stimulation of the same target brain tissue in the primary auditory cortex to reduce neuroplasticity effects associated with the therapeutic electrical stimulation. As another example, where therapeutic electrical stimulation of the auditory cortex is desired, one or more stimulation programs 36 may be set up for therapeutic electrical stimulation of target brain tissue in the primary auditory cortex and one or more other stimulation programs 36 may be set up for electrical stimulation of different target brain tissue in the primary auditory cortex or elsewhere in the brain to reduce neuroplasticity effects associated with the therapeutic electrical stimulation.

As described above, in one embodiment, the nature of the neuroplasticity reducing electrical stimulation may be varied more or less continually, whether in a predetermined or randomized manner, to prevent, delay, or otherwise reduce the ability of the brain to adapt to the neuroplasticity reducing electrical stimulation and dynamically reorganize itself accordingly. In a more particular embodiment, where the neuroplasticity reducing electrical stimulation is provided concurrently with therapeutic electrical stimulation, the neuroplasticity reducing electrical stimulation may be randomized or otherwise varied about the therapeutic electrical stimulation to achieve this result. In essence, the randomized or otherwise varied neuroplasticity reducing electrical stimulation makes it more difficult for the brain to dynamically reorganize itself to overcome the effects of the therapeutic electrical stimulation.

Although the present invention has been described primarily in connection with electrical stimulation to reduce neuroplasticity effects, the present invention contemplates electrical stimulation of the brain using two or more stimulation sets 30 for any suitable purposes. For example, electrical stimulation of the brain may be provided using two or more stimulation sets 30 for therapeutic purposes rather than, or independent of, neuroplasticity reducing purposes. Two or more stimulation sets 30 may be used to stimulate the same nerve tissue in two or more ways, to stimulate two or more locations using a single electrical stimulation lead 14, or otherwise.

The present invention contemplates any suitable circuitry within stimulation source 12 for generating and transmitting signals for electrical stimulation of a person's nerve tissue. Example circuitry which may be used is illustrated and described in U.S. Pat. No. 6,609,031 B1, which is hereby incorporated by reference herein as if fully illustrated and described herein. In certain embodiments, stimulation provided using such circuitry is to provide reduce neuroplasticity effects in the nerve tissue (whether the stimulation is provided independent of or concurrently with any electrical stimulation for therapeutic purposes). In other embodiments, as described in the preceding paragraph, stimulation provided using such circuitry may be to provide therapeutic effects (independent of any electrical stimulation that may be provided to reduce neuroplasticity effects), in this case preferably using two or more stimulation sets 30.

Although the present invention has been described above in connection with several embodiments, a plethora of changes, substitutions, variations, alterations, transformations, and modifications may be suggested to one skilled in the art, and it is intended that the present invention encompass such changes, substitutions, variations, alterations, transformations, and modifications as fall within the spirit and scope of the appended claims.

1. An electrical stimulation system providing reduced neuroplasticity effects in a person's brain, comprising:
an electrical stimulation lead adapted for implantation into the person's brain for electrical stimulation of target brain tissue, the lead comprising a plurality of electrodes adapted to be positioned near the target brain tissue and to deliver electrical stimulation energy to the target brain tissue;
a stimulation source connectable to the electrical stimulation lead and operable to generate signals for transmission to the electrodes of the electrical stimulation lead to cause the electrodes to deliver electrical stimulation energy to the target brain tissue to reduce neuroplasticity effects in the person's brain.

2. The system of claim 1, wherein the neuroplasticity reducing electrical stimulation is randomized to make it more difficult for the brain to adapt to the neuroplasticity reducing electrical stimulation and dynamically reorganize itself accordingly.

3. The system of claim 1, wherein the stimulation source is operable to generate signals for transmission to the electrodes to cause the electrodes to deliver electrical stimulation energy to the target brain tissue to provide therapeutic electrical stimulation of the target brain tissue in addition to neuroplasticity reducing electrical stimulation, the neuroplasticity reducing electrical stimulation making it more difficult for the brain to dynamically reorganize itself to overcome effects of the therapeutic electrical stimulation.

4. The system of claim 3, wherein the target brain tissue is located in the person's primary auditory cortex and the therapeutic electrical stimulation is provided to treat tinnitus.

5. The system of claim 3, wherein the stimulation source is operable to generate the signals for providing therapeutic electrical stimulation of the target brain tissue in association with the signals generated for reducing neuroplasticity effects, such that the electrodes are caused to deliver electrical energy for providing therapeutic electrical stimulation and electrical energy for reducing neuroplasticity effects substantially concurrently to the target brain tissue.

6. The system of claim 3, wherein the neuroplasticity reducing electrical stimulation is randomized about the therapeutic electrical stimulation.

7. The system of claim 3, wherein average intensity of the electrical stimulation energy delivered to reduce neuroplasticity effects is less than or equal to average intensity of the electrical stimulation energy delivered to provide therapeutic electrical stimulation.

8. The system of claim 1, wherein the stimulation source is operable to generate the signals according to one or more stimulation sets each specifying a plurality of stimulation parameters, the stimulation parameters for a stimulation set comprising a polarity for each electrode at each of one or more times within a stimulation pulse for the stimulation set.

9. The system of claim 8, wherein the polarity for at least one electrode changes for each of a sequence of times according to the stimulation parameters for the stimulation set.

10. The system of claim 8, wherein the polarity for an electrode at a time comprises either a relatively positive polarity, a relatively negative polarity, or an intermediate polarity between the relatively positive polarity and relatively negative polarity.

11. The system of claim 8, wherein the stimulation parameters for a stimulation set further comprise an amplitude, a frequency, phase information, and a pulse width for the stimulation pulse.

12. The system of claim 8, wherein at least one stimulation parameter for a stimulation set is randomized within a predetermined range during execution of the stimulation set.

13. The system of claim 8, wherein:
the stimulation source is operable to generate signals for transmission to the electrodes to cause the electrodes to deliver electrical stimulation energy to the target brain tissue to provide therapeutic electrical stimulation of the target brain tissue in addition to neuroplasticity reducing electrical stimulation, the neuroplasticity reducing electrical stimulation making it more difficult for the brain to dynamically reorganize itself to overcome effects of the therapeutic electrical stimulation; and
the stimulation source is operable to generate the signals according to one or more stimulation sets each specifying a plurality of stimulation parameters for a plurality of stimulation pulses, one or more of the stimulation pulses accomplishing therapeutic electrical stimulation of the target brain tissue and one or more other of the stimulation pulses accomplishing neuroplasticity reducing electrical stimulation of the target brain tissue.

14. The system of claim 13, wherein a therapeutic electrical stimulation pulse is separated from a successive therapeutic electrical stimulation pulse by a number of neuroplasticity reducing stimulation pulses greater than or equal to zero, the number being either predetermined or randomized.

15. The system of claim 8, wherein:
the stimulation source is operable to generate signals for transmission to the electrodes to cause the electrodes to deliver electrical stimulation energy to the target brain tissue to provide therapeutic electrical stimulation of the target brain tissue in addition to neuroplasticity reducing electrical stimulation, the neuroplasticity reducing electrical stimulation making it more difficult for the brain to dynamically reorganize itself to overcome effects of the therapeutic electrical stimulation; and
the stimulation source is operable to generate the signals according to a plurality of stimulation programs each comprising one or more stimulation sets, each stimulation set specifying a plurality of stimulation parameters, one or more of the stimulation sets accomplishing therapeutic electrical stimulation of the target brain tissue and one or more other of the stimulation sets accomplishing neuroplasticity reducing electrical stimulation of the target brain tissue.

16. The system of claim 15, wherein a therapeutic electrical stimulation set is separated from a successive therapeutic electrical stimulation set by a number of neuroplasticity reducing stimulation sets greater than or equal to zero, the number being either predetermined or randomized.

17. The system of claim 1, wherein the neuroplasticity effects are associated with therapeutic electrical stimulation of the person's brain or are due to previous injury or disease.

18. The system of claim 1, wherein the target brain tissue comprises brain tissue located in a region of the person's cortex.

19. The system of claim 18, wherein the target brain tissue comprises brain tissue associated with at least one of the person's:
primary motor cortex;
primary somatosensory cortex;
primary visual cortex; and
primary auditory cortex.

20. The system of claim 1, wherein the target brain tissue comprises brain tissue located in a region of the person's thalamus.

21. A method providing reduced neuroplasticity effects in a person's brain, comprising:
using a stimulation source to generate signals for transmission to electrodes of an electrical stimulation lead implanted in the person's brain to cause the electrodes to deliver electrical stimulation energy to target brain tissue to reduce neuroplasticity effects; and
in response to the signals transmitted from the stimulation source, using the electrodes of the electrical stimulation lead implanted in the person's brain to deliver electrical stimulation energy to the target brain tissue to reduce neuroplasticity effects in the person's brain.

22. The method of claim 21, wherein the neuroplasticity reducing electrical stimulation is randomized to make it more difficult for the brain to adapt to the neuroplasticity reducing electrical stimulation and dynamically reorganize itself accordingly.

23. The method of claim 21, further comprising using the stimulation source to generate signals for transmission to the electrodes to cause the electrodes to deliver electrical stimulation energy to the target brain tissue to provide therapeutic electrical stimulation of the target brain tissue in addition to neuroplasticity reducing electrical stimulation, the neuroplasticity reducing electrical stimulation making it more difficult for the brain to dynamically reorganize itself to overcome effects of the therapeutic electrical stimulation.

24. The method of claim 23, wherein the target brain tissue is located in the person's primary auditory cortex and the therapeutic electrical stimulation is provided to treat tinnitus.

25. The method of claim 24, wherein the stimulation source generates the signals for providing therapeutic electrical stimulation of the target brain tissue in association with the signals generated for reducing neuroplasticity effects, such that the electrodes deliver electrical energy for providing therapeutic electrical stimulation and electrical energy for reducing neuroplasticity effects substantially concurrently to the target brain tissue.

26. The method of claim 24, wherein the neuroplasticity reducing electrical stimulation is randomized about the therapeutic electrical stimulation.

27. The method of claim 24, wherein average intensity of the electrical stimulation energy delivered to reduce neuroplasticity effects is less than or equal to average intensity of the electrical stimulation energy delivered to provide therapeutic electrical stimulation.

28. The method of claim 21, wherein the stimulation source generates the signals according to one or more stimulation sets each specifying a plurality of stimulation parameters, the stimulation parameters for a stimulation set comprising a polarity for each electrode at each of one or more times within a stimulation pulse for the stimulation set.

29. The method of claim 28, wherein the polarity for at least one electrode changes for each of a sequence of times according to the stimulation parameters for the stimulation set.

30. The method of claim 28, wherein the polarity for an electrode at a time comprises either a relatively positive polarity, a relatively negative polarity, or an intermediate polarity between the relatively positive polarity and relatively negative polarity.

31. The method of claim 28, wherein the stimulation parameters for a stimulation set further comprise an amplitude, a frequency, phase information, and a pulse width for the stimulation pulse.

32. The method of claim 28, wherein at least one stimulation parameter for a stimulation set is randomized within a predetermined range during execution of the stimulation set.

33. The method of claim 28, wherein:
the stimulation source generates signals for transmission to the electrodes to cause the electrodes to deliver electrical stimulation energy to the target brain tissue to provide therapeutic electrical stimulation of the target brain tissue in addition to neuroplasticity reducing electrical stimulation, the neuroplasticity reducing electrical stimulation making it more difficult for the brain to dynamically reorganize itself to overcome effects of the therapeutic electrical stimulation; and
the stimulation source generates the signals according to one or more stimulation sets each specifying a plurality of stimulation parameters for a plurality of stimulation pulses, one or more of the stimulation pulses accomplishing therapeutic electrical stimulation of the target brain tissue and one or more other of the stimulation pulses accomplishing neuroplasticity reducing electrical stimulation of the target brain tissue.

34. The method of claim 33, wherein a therapeutic electrical stimulation pulse is separated from a successive therapeutic electrical stimulation pulse by a number of neuroplasticity reducing stimulation pulses greater than or equal to zero, the number being either predetermined or randomized.

35. The system of claim 28, wherein:
the stimulation source generates signals for transmission to the electrodes to cause the electrodes to deliver electrical stimulation energy to the target brain tissue to provide therapeutic electrical stimulation of the target brain tissue in addition to neuroplasticity reducing electrical stimulation, the neuroplasticity reducing electrical stimulation making it more difficult for the brain to dynamically reorganize itself to overcome effects of the therapeutic electrical stimulation; and
the stimulation source generates the signals according to a plurality of stimulation programs each comprising one or more stimulation sets, each stimulation set specifying a plurality of stimulation parameters, one or more of the stimulation sets accomplishing therapeutic electrical stimulation of the target brain tissue and one or more other of the stimulation sets accomplishing neuroplasticity reducing electrical stimulation of the target brain tissue.

36. The method of claim 35, wherein a therapeutic electrical stimulation set is separated from a successive therapeutic electrical stimulation set by a number of neuroplasticity reducing stimulation sets greater than or equal to zero, the number being either predetermined or randomized.

37. The method of claim 21, wherein the neuroplasticity effects are associated with therapeutic electrical stimulation of the person's brain or are due to previous injury or disease.

38. The method of claim 21, wherein the target brain tissue comprises brain tissue located in a region of the person's cortex.

39. The method of claim 38, wherein the target brain tissue comprises brain tissue associated with at least one of the person's:
primary motor cortex;
primary somatosensory cortex;
primary visual cortex; and
primary auditory cortex.

40. The method of claim 21, wherein the target brain tissue comprises brain tissue located in a region of the person's thalamus.

41. An electrical stimulation system providing electrical stimulation of a person's brain to reduce neuroplasticity effects associated with concurrent therapeutic electrical stimulation of the person's brain, comprising:
an electrical stimulation lead adapted for implantation into the person's brain for electrical stimulation of target brain tissue, the lead comprising a plurality of electrodes adapted to be positioned near the target brain tissue and to deliver electrical stimulation energy to the target brain tissue in response to received signals; and
a stimulation source adapted for implantation into the person's body and operable to concurrently:
generate signals for transmission to the electrodes of the lead to cause the electrodes to deliver electrical stimulation energy to the target brain tissue within the stimulation pulse to provide therapeutic electrical stimulation of the target brain tissue; and
generate signals for transmission to the electrodes of the lead to cause the electrodes to deliver electrical stimulation energy to the target brain tissue within the stimulation pulse to reduce neuroplasticity effects associated with the therapeutic electrical stimulation of the target brain tissue, the neuroplasticity reducing electrical stimulation being randomized about the therapeutic electrical stimulation, the neuroplasticity reducing electrical stimulation making it more difficult for the brain to dynamically reorganize itself to overcome effects of the therapeutic electrical stimulation;
the signals generated according to a plurality of stimulation programs each comprising one or more stimulation sets, each stimulation set specifying a plurality of stimulation parameters comprising a polarity for each electrode of the lead at each of one or more times, the polarities for the electrodes changing over time according to the stimulation parameters, one or more stimulation sets accomplishing therapeutic electrical stimulation of the target brain tissue and one or more other stimulation sets accomplishing neuroplasticity reducing electrical stimulation of the target brain tissue, each therapeutic electrical stimulation set being separated from a next therapeutic electrical stimulation set by a number of neuroplasticity reducing stimulation sets that is greater than or equal to zero.

42. An electrical stimulation system providing reduced neuroplasticity effects in a person's nerve tissue, comprising:
an electrical stimulation lead adapted for implantation into the person's body for electrical stimulation of target nerve tissue, the lead comprising a plurality of electrodes adapted to be positioned near the target nerve tissue and deliver electrical stimulation energy to the target nerve tissue;
a stimulation source connectable to the electrical stimulation lead and operable to generate signals for transmission to the electrodes of the electrical stimulation lead to cause the electrodes to deliver electrical stimulation energy to the target nerve tissue to reduce neuroplasticity effects in the target nerve tissue.

43. The system of claim 42, wherein the target nerve tissue comprises one of brain tissue, spinal cord tissue, and peripheral nerve tissue.

44. An electrical stimulation system for stimulating a person's brain, comprising:
an electrical stimulation lead adapted for implantation into the person's brain for electrical stimulation of target brain tissue, the lead comprising a plurality of electrodes adapted to be positioned near the target brain tissue and to deliver electrical stimulation energy to the target brain tissue;
two or more stimulation sets each specifying stimulation parameters; and
a stimulation source connectable to the electrical stimulation lead and operable to generate signals according to the two or more stimulation sets for transmission to the electrodes of the electrical stimulation lead to cause the electrodes to deliver electrical stimulation energy to the target brain tissue.

45. The system of claim 44, wherein the stimulation occurs according to a first stimulation set in one or more first stimulation periods and according to a second stimulation set in one or more second time periods distinct from the one or more first time periods.
